(19) United States
(12) Patent Application Publication
(10) Pub. No.: US 2023/0293095 A1
(43) Pub. Date: Sep. 21, 2023

(54) A WIRELESS WEARABLE VOICE MONITORING SYSTEM

(71) Applicants: UNIVERSIDAD TÉCNICA FEDERICO SANTA MARÍA, Valparaíso (CL); UNIVERSIDAD DE VALPARAÍSO, Valparaíso (CL)

(72) Inventors: Matías ZAÑARTU SALAS, Viña del Mar (CL); Alejandro José WEINSTEIN OPPENHEIMER, Valparaíso (CL); Johannes Bruno SCHWARZENBERG OLIVARES, Viña del Mar (CL); Gonzalo Ariel CARRASCO REYES, Viña del Mar (CL); Javier Ignacio ROSAS SOTO, Valparaíso (CL); Fabián Vicente RUBILAR JAMÉN, Viña del Mar (CL)

(21) Appl. No.: 18/019,784

(22) PCT Filed: Aug. 5, 2021
(86) PCT No.: PCT/IB2021/057224

§ 371 Date: Feb. 3, 2023

Related U.S. Application Data
(60) Provisional application No. 63/061,348, filed on Aug. 5, 2020.

(51) Int. Cl. A61B 5/00; G10L 25/66; G10L 25/84; H04R 1/46; H04R 3/04; G06T 11/20

(52) U.S. Cl. CPC A61B 5/4803; G10L 25/66; G10L 25/84; H04R 1/46; H04R 3/04; G06T 11/206; A61B 5/6822; A61B 5/0002; H04R 2420/07; A61B 2562/0219; A61B 5/7475; A61B 5/742

(57) ABSTRACT

A wearable voice detection system in the form of a necklace is disclosed, which allows to monitor the use of the voice in everyday conditions of use by means of an autonomous operation, and to maintain at the same time the accuracy and integrity of the signals obtained. The system comprises: a sensor device comprising a sound detection means and an accelerometer registering sound signals and acceleration variations in the skin of a user; a control device in electrical communication with the sensor device, the control device comprising processing means and data transmission means; wherein the control device is configured to receive and process the signals obtained by the sensor device and to transmit processed data to an external location.

### CROSS REFERENCES TO RELATED APPLICATIONS

This application claims the benefit of Applicants' prior provisional application, number U.S. 63/061,348, filed on Aug. 5, 2020.

### BACKGROUND

The voice is a fundamental tool in people's lives as communication of a person is mainly conditioned by the voice. Therefore, the appearance of conditions or voice disorders affecting or canceling the ability to speak involves a significant decrease in quality of life and a serious occupational health problem. Vocal folds nodules, muscle dysphonia, tension, spasmodic dysphonia, paralysis of the folds, or temporary loss of voice, among others, annually affect millions of people worldwide.

Currently, treatments for voice pathologies or disorders are only carried out through visits to medical consultations and in limited times, which does not allow obtaining constant and sufficient information to adequately describe its daily use. In this sense, clinical monitoring is essential for the effectiveness of the diagnosis and treatment of these pathologies, but its implementation is limited to medical consultations, which excludes the possibility of obtaining information that adequately describe the actual use of the voice. Without this information, specialists can only design broad-spectrum therapies, which are often inefficient and result in a high number of recurrences.

Recently, to provide a proper monitoring of patients with voice conditions, wearable devices have been developed, commonly in the form of a necklaces, that allows monitoring the use of the voice using electrodes, microphones, and other equipment.

An exemplary technology of the prior art is described in document US 2014/235977 A1, which discloses a neck-worn sensor that is a single, body-worn system that measures several parameters from an ambulatory patient. From stroke volume, a first algorithm employing a linear model can estimate the patient's pulse pressure. And from pulse pressure and pulse transit time, a second algorithm, also employing a linear algorithm, can estimate systolic blood pressure and diastolic blood pressure. Thus, the necklace can measure all five vital signs along with hemodynamic parameters. It also includes a motion-detecting accelerometer, from which it can determine motion-related parameters such as posture, degree of motion, activity level, respiratory-induced heaving of the chest, and falls.

The prior art device described above addresses the problems related to the portability of the equipment, allowing a semi-continuous monitoring of the user by means of a wearable device. However, the configuration of this device is intended for general data logging, but there is no need for extra care with the signal integrity. This is clear considering the purpose of the transducer used, an accelerometer, which aims to record the user movement, but there are no further treatments to thoroughly analyze the characteristics of the voice signals.

Another approach to register and analyze voice signals is based in the use of neck-surface accelerometers, which allow for non-invasive and non-obstructive speech and swallow measures. One document that represents this kind of technologies of the prior art is document US 2014/0066724 A1, which describes a system and method to assess vocal function of a subject. This system includes an accelerometer configured to acquire surface acceleration data associated with vocal functionality of the subject and a computer system configured to analyze the surface acceleration data and to estimate glottal airflow wave-forms produced by the subject based on the surface acceleration data. The computer system performs the analysis and estimation by applying an inverse filter to the surface acceleration data based on a calibrated transmission line model and generates an indication of vocal functionality of the subject based on the estimated glottal airflow waveforms.

However, the system described above requires the connection of a sensor to a computer system to analyze the data obtained by the sensor. Accordingly, the system described do not address the portability issues and do not comply with the requirements for an autonomous operation. Additionally, the computer system requires the use of audio codecs to pre-process the data and store it digitally. This pre-process may vary considering the computer system and may include gain, band-pass filtering and noise reduction that will cause distortion of the signal, thereby affecting the integrity of the signal and the subsequent analysis of the data. Also, being a wired device it's prone to have unintended connection problems.

Accordingly, there is a need of providing an autonomous device to record the complete use of the voice during the day in everyday conditions, and able to maintain at the same time the accuracy and integrity of the signal, so the data can be used to obtain and estimate certain parameters and indicators that are useful for the assessment of the vocal function.

### BRIEF SUMMARY OF THE INVENTION

The invention refers to a wearable voice detection system, in the form of a necklace, that allows monitoring the use of the voice of a user, the system comprising:
a sensor device comprising a sound detection means and an accelerometer registering sound signals and acceleration variations in the skin of a user; and
a control device in electrical communication with the sensor device, the control device comprising processing means and data transmission means;
   wherein the control device is configured to receive and process the signals obtained by the sensor device and to transmit processed data to an external location.

The system described comprises compact, small-sized elements that allow portability and usability as a wearable object, thereby allowing a continuous monitoring of the voice in everyday conditions of use. By the combined use of the sound detection means and the accelerometer, the system is capable of accurately registering and analyzing the use of the vocal folds in order to estimate a series of physiological parameters that are not only of clinical interest to researchers and voice professionals, but also to any professional who uses their voice as a work tool and requires precise monitoring of the health of their voice, such as announcers, singers, teachers, journalists, among others. Thus, the specific configuration of the system described above is capable of substantially improving the evaluation, diagnosis, and monitoring capacity of vocal pathologies that affect millions of people a year in the world, which, in their most serious cases, even lead to permanent loss of voice.

Therefore, the operation of the device of the present invention is based in the use of an accelerometer and a microphone simultaneously, allowing to process signals in real time to deliver instant feedback to users based on their vocal use, which constitutes a disruptive methodology for vocal therapies.

One of the key features of the system described is that the processing means are separate from the sensing device, thus providing separate, small, and compact elements which facilitates the wearability and comfort for the user, by providing a minimally invasive wearable device in the form of a necklace.

Preferably, the processed data obtained from the captured signals can be stored in a data storage means in the control device, and can be transmitted periodically or in real time to an external location, for example, an external computer or in the cloud, where it can be later analyzed by a specialist, in a post-processing or medical analysis. Alternatively, the control device can be configured to transmit information in real time to a user interface, such as an app in a smartphone via Bluetooth.

One of the key aspects that are addressed by the invention is the integrity of the signal. This is accomplished on one hand by selecting the correct transducers, an accelerometer with the specific bandwidth for vocal applications combined with a microphone, and on the other hand, by the optimal data acquisition process, which includes filtering by hardware to precondition de signal and then the use of an audio codec to further process the signals. The combination of this characteristics of the invention provides full control over the behavior of the input signal, minimizing phase and harmonic distortions and, finally, encoding de data for transfer and storage.

More particularly, the use of an accelerometer strategically positioned on the trachea allows the estimation of glottic flow, subglottic pressure, and other determining variables for the identification of vocal hyperfunction. Additionally, the data obtained by the accelerometer is complemented by an environmental capture of sound by the sound detecting means, which can be selectively controlled to be turned on and off, thereby allowing the patient to decide when he do not want certain information to be recorded. The combination of both mechanisms allows the instant detection of vocal abuse.

The combined and simultaneous use of two kind of signals, sound, and acceleration, allows the delivery of clinically relevant information for the evaluation of vocal function and has been shown to better identify patterns of vocal abuse, generating a more useful device and, therefore, having greater appreciation by health professionals and patients. Its features allow feedback using advanced parameters and indicators for vocal use, which is a revolutionary therapeutic methodology, performing a pre-processing of the signals and transmitting data to provide feedback to the user. In addition, its wireless, ergonomic, and discreet design conceals its medical nature and makes it easy to use, providing an object that does not look like a medical device, allowing the user to use the device as a wearable item without affecting the quality of the captured signals.

### BRIEF DESCRIPTION OF THE DRAWINGS

FIGS. 1A and 1B illustrates preferred embodiments of the wearable voice detection system of the present invention.

FIG. 2 illustrates an exploded view of the sensor device in a preferred embodiment of the invention.

FIG. 3 illustrates an exploded view of the control device in a preferred embodiment of the invention.

### DETAILED DESCRIPTION AND BEST MODE OF IMPLEMENTATION

The following is a detailed description of exemplary embodiments to illustrate the principles of the invention. The embodiments are provided to illustrate aspects of the invention, but the invention is not limited to any embodiment. The scope of the invention encompasses numerous alternatives, modifications and equivalent, only limited by the embodiments of the claims.

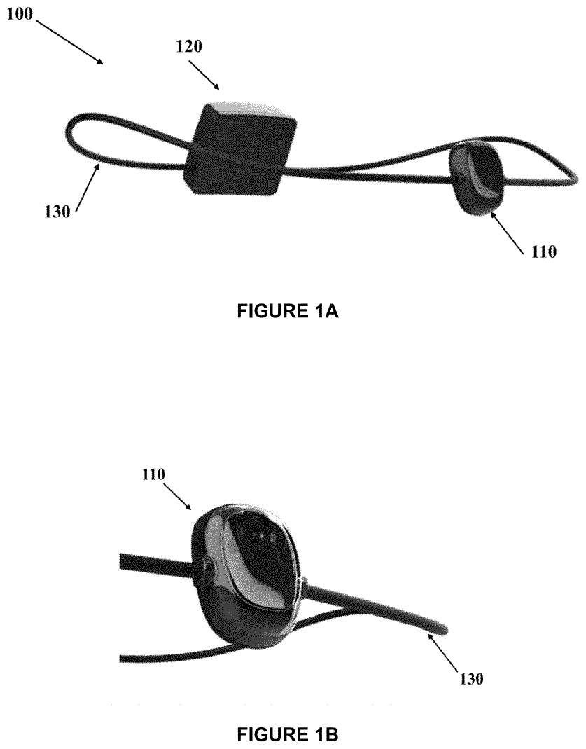

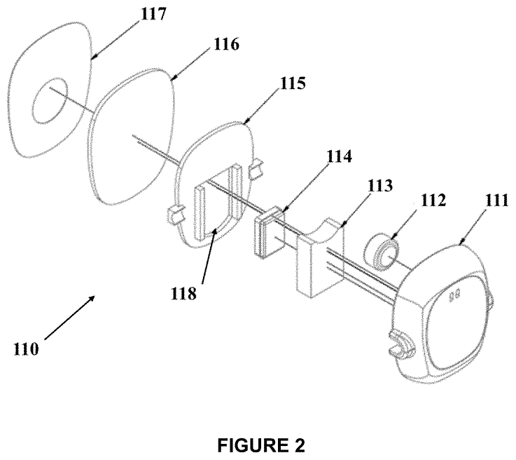

According to FIGS. 1A, 1B and 2, in a first aspect of the invention a wearable voice detection system (100) in the form of a necklace is disclosed, the system comprising:
a sensor device (110) comprising a sound detection means (112) and an accelerometer (114) registering sound signals and acceleration variations in the skin of a user;
a control device (120) in electrical communication with the sensor device (110), the control device comprising processing means and data transmission means;
   wherein the control device is configured to receive and process the signals obtained by the sensor device and to transmit processed data to an external location.

As disclosed in FIGS. 1A and 1B, preferably the control device and the sensor device are connected by means of an electrical connection (130), which allows the communication between both elements to allow the transfer of the signals captured by the sensor device (110) to the control device (120) to be processed. This configuration of separated elements allows to achieve small and compact sensor and control devices, thus providing a comfortable, non-invasive system for the user. More preferably, the system (100) is configured to locate the control device (120) on the back of the neck and the sensor device (110) in the frontal area, close to the trachea. More preferably, the sensor device (110) locates on the neck skin between the sternal notch and the thyroid prominence, to allow a more accurate reception of the signals.

Referring to FIG. 2, an exploded view of a preferred embodiment of the sensor device (110) is disclosed. In this embodiment, the sensor device (110) comprises a front casing (111), the sound detecting means (112), an accelerometer housing (113), the accelerometer (114), a back cover (115), adhesive means (117) and a rubber or silicone pad (116). The front casing (111) and the back cover (115) are configured to couple and provide a housing for the sound detecting means (112) and the accelerometer (114). The back cover (115) can include a hole (118) to allow a communication between the accelerometer (114) and skin of the user. The adhesive means (117) are configured to allow a removable fixation of the sensor device (110) in the skin of the user, preferably by means of a double contact tape. Additionally, the elements of the sensor device are preferably design and selected so that they do not affect the capture of the signals, especially the back cover (115), the rubber pad (116) and the adhesive means (117), wherein the adhesive means (117) must allow a fixation able to permit the transmission of vibrations for the proper operation of the accelerometer.

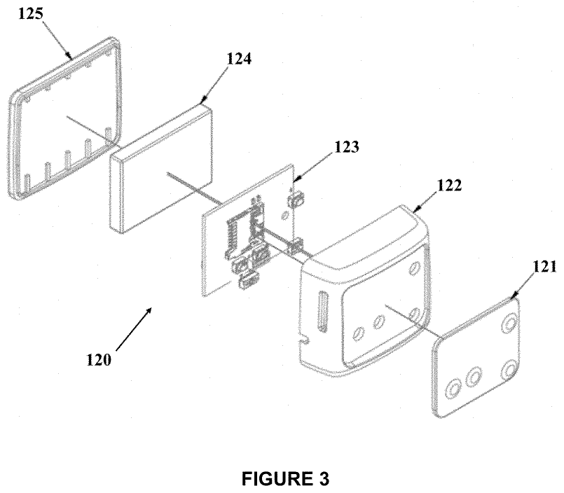

Referring to FIG. 3, an exploded view of a preferred embodiment of the control device (120) is disclosed. In this embodiment, the control device (120) comprises a control means (121), front casing (122), the processing means (123), energy storage means (124) and a back cover (125). The control means (121) is configured to include one or more buttons to allow a control of some operational features of the system. Preferably, the energy storage means (124) is configured to provide a lifetime of more than 12 hours of continuous recording, thus allowing uninterrupted monitoring for a whole day, obtaining measurements over several days.

The energy storage means (124) allows an autonomous operation of the system. To accomplish this operation, the energy storage means (124) preferably consist of a battery that allows the system to operate without a physical connection to an external source. In this embodiment, a charging port can be included in the control device to allow the charge of the battery with an external source.

The processing means (123) is configured to implement voice processing algorithms and to command all the elements of the system (100). Preferably, the processing means (123) consist of a printed circuit board, configured with state-of-the-art electronic technology, and it is capable of processing signals to deliver instant feedback (biofeedback) to users based on their vocal use, which is a new methodology for vocal therapies. The data obtained can be stored and processed safely in the cloud (HIPPA compliant) or an external location, thanks to unique algorithms specially designed for the interpretation of this data, which allows the generation of new useful information for health professionals (such as flow glottic airway, subglottic pressure, and vocal efficiency).

In preferred embodiments of the invention, the processing means (123) include data storage means configured to store all the data that is being processed by the system, thus allowing to process data while the device is be used.

The control device preferable include data transmission means configured to allow the transmission of the processed data to an external location. Preferably, the transmission means is configured to transmit the processed data periodically or in real time to the external location, for example, an external computer or in the cloud, where it can be later analyzed by a specialist, in a post-processing or medical analysis. Alternatively, the control device can be configured to transmit information in real time to a user interface, such as a computer or an app in a smartphone via Bluetooth.

In preferred embodiments, the processed data is preferably transmitted to a user interface, which is configured to visualize and analyze the data in a corresponding software, aimed at researchers and voice professionals. The processed data can be visualized in all kinds of mobile platforms and computers, both for health professionals and patients, to visualize and analyze information processed periodically or in in real time, and to track unprecedented vocal function.

The control means (121) can consist in a keypad including one or more buttons, or can be configured as a touchpad, and is configured to provide basic commands for the operation of the system, such as turning the system on and off, or other alternative functions. Additionally, the control means can include displaying means, such as a screen or lights to provide basic information about the status of the operation, like the battery level, or other operation features.

In preferred embodiments, the processing means is configured to implement several treatments or algorithms to the input signal, including filtering by hardware to precondition de signal and then the use of an audio codec to further process the signals. This procedure in combination with the use of the correct transducers, an accelerometer with the specific bandwidth for vocal applications combined with a microphone, allow to maintain the integrity of the signals, providing full control over the behavior of the input signal, minimizing phase and harmonic distortions and, finally, encoding de data for transfer and storage.

The processing means is configured to implement a vocal analysis engine, which is the core analysis obtained using the system (100). The vocal analysis engine comprises several algorithms designed for the assessment of the vocal function, with two analysis modules that operates with a neck surface acceleration signal (ACC) and a sound signal obtained by the sound detecting means, preferably a microphone (MIC).

The first analysis module is the “standard vocal health analysis”, which considers speech signal processing approaches that are not available in any prior ambulatory voice monitor. The following features are included in this module:
MIC signal de-intelligibility, in which the high-bandwidth signal is transformed into selected features, such as SPL (Sound Pressure Level) via MIC RMS (Root Mean Squared), magnitude of FFT (Fast Fourier Transform).
Daily ACC placement calibration check, which is made via both MIC RMS and ACC data after VAD (vocal activity detection).
Robust vocal activity detection (VAD) on the ACC signal and related VAD features, using ACC and MIC correlation.
Vocal intensity that is made via both MIC RMS and ACC data after VAD.
Fundamental frequency (f0), from the ACC signal using autocorrelation.
Vocal Dose (SPL and f0 from the ACC signal), including cycle and distance dose.
Acoustic dosimeter, including a background noise level detection via VAD and MIC signal processing.
Vocal Efficiency (SPL vs ACC).
H1-H2, ratio between the first and the second harmonic, FFT base on ACC signal.
Spectral tilt, High resolution filtering on FFT of ACC signal.
CPP (Cepstral peak prominence) on ACC and MIC signals.

Additionally, the following advanced features can be obtained by the processing means, which have been shown to better identify vocal hyperfunctional behaviors and are key for a more comprehensive assessment of vocal function in an ambulatory scenario:
Aerodynamic features like AC flow (unsteady flow of air), MFDR (maximum flow declination rate), OQ (Open quotient, ratio of the open period to the entire glottal cycle's duration), SQ (speed quotient, ratio between the opening and the closing phase of the vocal folds) obtained via the IBIF (Impedance-Based Inverse Filtering) algorithm from the ACC signal, OVV (oral airflow volume velocity), subglottic pressure. This also includes a calibration scheme to obtain robust subject-specific IBIF parameters using MIC inverse filtering, instead of the OVV (oral airflow volume velocity) signal (obtained by using specialized equipment and in controlled environment) in the original IBIF algorithm). IBIF model parameters are obtained using a weighting method that combines information from the estimation from different vowels. This new calibration scheme has not been reported before in the scientific/technical literature.
Subglottal pressure is obtained using multivariate linear regression (using the prior aerodynamic features, ACC and IBIF features) using SPL from the MIC signal.
Singing detection using both ACC and MIC signal.

Therefore, by means of the features described above the invention allows to obtain and estimate parameters and indicators that are useful for the assessment of the vocal function, such as SPL, VAD, f0, H1-H2, CPP, which nowadays can only be obtained in clinical facilities, and some of them, such as the aerodynamics features, requires highly invasive procedures to be obtained. The voice detection system described herein allows to obtain these parameters and indicators in a continuous operation by means of a portable device.

Additionally, the processing means is configured to provide daily reports. Once the vocal health indicators are calculated, the Vocal Analysis Engine generates a summary of the results. These results are saved and sent to both the users, for example via a mobile application or a web browser, and the Health Specialist. The specific content includes raw features, daily/weekly statistics, and daily biofeedback summary. As complement for the daily reports, the Vocal Analysis Engine is also capable of generating graphic information based on the daily reports and user-requested analyses. Features in this module can be selected as desired and include:
Waveform and spectral visualization across time with user defined window time.
Multiple vocal health measures across time with smoothing and user defined window time.
Uni- and bi-dimensional histograms for any of the standard or advanced vocal measures.
Visualization with the UMAP dimensionality reduction technique.
A comparison of the parameters in the same time window between the different days of the analysis.
Correlate alterations in the parameters obtained with the user's habits (smoking, eating, screaming, etc.) and environmental variables.
Obtaining vocal efficiency level indicators, which correspond to indicators that describe a “voice quality”. These indicators allow the patients to notice their improvement.
Estimating parameters to identify and support the diagnosis of different pathologies and/or health conditions, even beyond the voice, such as for example Parkinson's.

While the present invention has been described in terms of particular embodiments and applications, in both summarized and detailed forms, it is not intended that these descriptions in any way limit its scope to any such embodiments and applications, and it will be understood that many substitutions, changes and variations in the described embodiments, applications and details of the methods and system illustrated herein can be made by those skilled in the art without departing from the spirit of this invention.

1. A wearable voice detection system (100) in the form of a necklace comprising:
a sensor device (110) comprising a sound detection means (112) and an accelerometer (114) registering sound signals and acceleration variations in the skin of a user;
a control device (120) in electrical communication with the sensor device (110), the control device comprising processing means and data transmission means;
wherein the control device is configured to receive and process the signals obtained by the sensor device and to transmit processed data to an external location.

2. A wearable voice detection system according to claim 1, wherein the control device and the sensor device are connected by means of an electrical connection (130) that allows the transfer of the signals captured by the sensor device (110) to the control device (120) to be processed.

3. A wearable voice detection system according to claim 1, wherein the control device (120) is located on the back of the neck and the sensor device (110) in the frontal area, close to the trachea to allow a more accurate reception of the signals.

4. A wearable voice detection system according to claim 3, wherein the sensor device (110) locates on the neck skin between the sternal notch and the thyroid prominence.

5. A wearable voice detection system according to claim 1, wherein the sensor device (110) comprises the sound detecting means (112), an accelerometer housing (113), the accelerometer (114), a front casing (111) and back cover (115) configured to couple and provide a housing for the sensor device.

6. A wearable voice detection system according to claim 5, wherein the sensor device (110) further comprises adhesive means (117) configured to allow a removable fixation of the sensor device (110) in the skin of the user, and a rubber or silicone pad (116) selected such to not affect the capture of the signals.

7. A wearable voice detection system according to claim 5, wherein the back cover (115) includes a hole (118) to allow a communication between the accelerometer (114) and skin of the user.

8. A wearable voice detection system according to claim 1, wherein the control device (120) comprises a control means (121), front casing (122), the processing means (123), energy storage means (124) and a back cover (125).

9. A wearable voice detection system according to claim 8, wherein the control means (121) includes one or more buttons to allow the control of some operational features of the system.

10. A wearable voice detection system according to claim 8, wherein the control means (121) includes a keypad having one or more buttons or a touchpad, and is configured to provide basic commands for the operation of the system, such as turning the system on and off, among others.

11. A wearable voice detection system according to claim 8, wherein the control means includes displaying means, such as a screen or lights to provide basic information about the status of the operation, like the battery level, or other operation features.

12. A wearable voice detection system according to claim 8, wherein the energy storage means (124) is configured to provide an autonomous operation of more than 12 hours for continuous recording.

13. A wearable voice detection system according to claim 1, wherein the processing means (123) is configured to process and deliver the signals to an external location.

14. A wearable voice detection system according to claim 1, wherein the processing means (123) include data storage means configured to storage all the data that is being processed by the system.

15. A wearable voice detection system according to claim 1, wherein the transmission means is configured to transmit the processed data to the external location, where it can be later analyzed by a specialist, in a post-processing or medical analysis.

16. A wearable voice detection system according to claim 15, wherein the processed data is preferably transmitted to a user interface, which is configured to visualize and analyze the data in a corresponding software.

17. A wearable voice detection system according to claim 1, wherein the processing means is configured to implement treatments or algorithms to the input signal, including filtering by hardware to precondition de signal and then the use of an audio codec to further process the signals.

18. A wearable voice detection system according to claim 1, wherein the processing means is configured to implement a vocal analysis engine, comprising algorithms designed for the assessment of the vocal function, with an analysis module that operates with a neck surface acceleration signal (ACC) and a sound signal obtained by the sound detecting means, preferably a microphone (MIC).

19. A wearable voice detection system according to claim 18, wherein the analysis module includes:
MIC signal de-intelligibility, in which the high-bandwidth signal is transformed into selected features, such as SPL (Sound Pressure Level) via MIC RMS (Root Mean Squared), magnitude of FFT (Fast Fourier Transform);
daily ACC placement calibration check, which is made via both MIC RMS and ACC data after VAD (vocal activity detection);
robust vocal activity detection (VAD) on the ACC signal and related VAD features, using ACC and MIC correlation;
vocal intensity that is made via both MIC RMS and ACC data after VAD;
fundamental frequency (f0), from the ACC signal using autocorrelation;
vocal dose (SPL and f0 from the ACC signal), including cycle and distance dose;
acoustic dosimeter, including a background noise level detection via VAD and MIC signal processing;
vocal efficiency (SPL vs ACC);
H1-H2, ratio between the first and the second harmonic, FFT base on ACC signal;
spectral tilt, High resolution filtering on FFT of ACC signal; and
CPP (Cepstral peak prominence) on ACC and MIC signals.

20. A wearable voice detection system according to claim 18, wherein the vocal analysis engine further comprises advanced features directed to better identify vocal hyperfunctional behaviors, including:
aerodynamic features like AC flow (unsteady flow of air), MFDR (maximum flow declination rate), OQ (Open quotient, ratio of the open period to the entire glottal cycle's duration), SQ (speed quotient, ratio between the opening and the closing phase of the vocal folds) obtained via the IBIF (Impedance-Based Inverse Filtering) algorithm from the ACC signal, including a calibration scheme to obtain robust subject-specific IBIF parameters using MIC inverse filtering;
subglottal pressure obtained by using multivariate linear regression (using the prior aerodynamic features, ACC and IBIF features) using SPL from the MIC signal; and
singing detection using both ACC and MIC signal.

21. A wearable voice detection system according to claim 18, wherein the processing means is configured to provide daily reports including data generated by the Vocal Analysis Engine, such as raw features, daily/weekly statistics, and daily biofeedback summary.

22. A wearable voice detection system according to claim 21, wherein the Vocal Analysis Engine is also capable of generating graphic information based on the daily reports and user-requested analyses, and provide a correlation between the obtained parameters and habits of the user and environmental characteristics, including:
waveform and spectral visualization across time with user defined window time;
multiple vocal health measures across time with smoothing and user defined window time;
uni- and bi-dimensional histograms for any of the standard or advanced vocal measures; and
visualization with the UMAP dimensionality reduction technique;
a comparison of the parameters in the same time window between the different days of the analysis.
correlate alterations in the parameters obtained with the user's habits (smoking, eating, screaming, etc.) and environmental variables.
obtaining vocal efficiency level indicators, which correspond to indicators that describe a “voice quality”. These indicators allow the patients to notice their improvement.
estimating parameters to identify and support the diagnosis of different pathologies and/or health conditions, even beyond the voice, such as for example Parkinson's.
